(19) United States
(12) Patent Application Publication
(10) Pub. No.: US 2022/0273903 A1
(43) Pub. Date: Sep. 1, 2022

(54) FAST ACTING HUMIDIFIER

(71) Applicant: FISHER & PAYKEL HEALTHCARE LIMITED, Auckland (NZ)

(72) Inventors: Guohua BAO, Auckland (NZ); Mark Samuel HAMILTON, Auckland (NZ); Adam John DARBY, Auckland (NZ); Johannes Nicolaas BOTHMA, Auckland (NZ)

(21) Appl. No.: 17/630,873

(22) PCT Filed: Aug. 5, 2020
(86) PCT No.: PCT/IB2020/057376

§ 371 Date: Jan. 27, 2022

Related U.S. Application Data
(60) Provisional application No. 62/882,985, filed on Aug. 5, 2019.

(51) Int. Cl. A61M 16/10

(52) U.S. Cl. CPC A61M 16/109; A61M 16/1095; A61M 2205/0211; A61M 2205/3317; A61M 2205/3368; A61M 2205/362

(57) ABSTRACT

Described herein is a humidifier comprising: a gas flow path, and a heater in the gas flow path. The heater comprises a heater element, and a heat conductor in thermal contact with the heating element. The heat conductor is foraminous.

### FIELD

The present disclosure relates to humidifiers for providing a flow of humidified gas to a patient.

### BACKGROUND

Humidifiers (when used for medical purposes (such as respiratory humidifiers or surgical humidifiers for example)) provide a flow of humidified gas to a patient. The humidification provided by the humidifier may in itself be used for providing treatment. Alternatively, the respiratory humidifier may be a supplementary add-on that humidifies a flow of gas produced by another medical device (e.g. positive airway pressure therapy, high flow therapy, or the like) used to provide respiratory treatment to the patient.

One type of humidifier is the heated humidifier, which humidifies a flow of gas passed over a heated volume of water, as shown in FIG. 1. A heated humidifier 1 typically comprises a humidification chamber 2 with liquid water 3, and a heater plate 4 that provides a source of thermal energy for the humidification chamber 2. The humidification chamber 2 has a heater base 5 that is heatable by a heater plate 4. The heater base 5 conducts thermal energy to the water 3 in the humidification chamber 2. The humidification chamber 2 has an inlet 6, that receives an incoming flow of gas 7, and the humidification chamber 2 has an outlet 8 that allows heated humidified flow of gas to leave the heated humidifier 9.

### SUMMARY

It is an object of the present disclosure to provide an apparatus and/or method for humidifying a flow of gas to a patient.

In one aspect, the present disclosure includes a humidifier, comprising: a gas flow path, a heater in the gas flow path, the heater comprising: a heater element, and a heat conductor in thermal contact with the heating element, wherein the heat conductor is foraminous.

The foraminous heat conductor may have voids in a solid structure.

This foraminous heat conductor may be foam.

The foam may be open cell foam.

The foam may be formed from metal or ceramic.

The foraminous heat conductor may comprise metal foam or ceramic foam.

The foraminous heat conductor may be for ameliorating the Leidenfrost effect.

The heat conductor may be for increasing the heat transfer rate.

The heater element may comprise PTC material.

The heater may be located within a conduit.

The humidifier may have an overflow drain.

The humidifier may further comprise a fluid delivery apparatus.

The fluid delivery apparatus may comprise a fluid reservoir.

The fluid delivery apparatus may be directly coupled to the heat conductor.

The fluid delivery apparatus may be adapted to drip fluid onto the heat conductor.

The heater may be in a humidifier tub.

The humidifier may be configured to operate at an operating temperature above about 20° C.

The humidifier may be configured to operate in a temperature range between about 60° C. and about 200° C., preferably between about 120° C. and about 150° C.

The heater element may comprise PTC material, and the foraminous heat conductor may comprise one or more of:
nickel
ceramic
copper
stainless steel.

The humidifier may be configured to operate in a temperature range from about 100° C. to about 300° C.

The humidifier may comprise a PTC heater and/or PTC heater element configured to operate in a temperature range from about 20° C. to about 300° C.

The PTC heater and/or PTC heater element may be configured to operate in a first and/or second configuration:
wherein in the first configuration the PTC heater and/or PTC heater element is configured to operate in a first temperature range from about 20° C. to about 100° C., the PTC heater and/or PTC-heater element in thermal contact with a first foraminous heat conductor.
wherein in the second configuration the PTC heater and/or PTC heater element is configured to operate in a second temperature range from about 100° C. to about 300° C., the PTC heater and/or PTC heater element in thermal contact with a second foraminous heat conductor.

The humidifier may comprise a non-PTC heater and/or non-PTC heater element configured to operate in a temperature range from about 20° C. to about 500° C.

The non-PTC heater and/or non-PTC heater element may be configured to operate in a first and/or second configuration:
wherein in the first configuration the non-PTC heater and/or non-PTC heater element is configured to operate in a first temperature range from about 20° C. to about 100° C., the non-PTC heater and/or non PTC-heater element in thermal contact with a first foraminous heat conductor.
wherein in the second configuration the non-PTC heater and/or non-PTC heater element is configured to operate in a second temperature range from about 100° C. to about 500° C., the non-PTC heater and/or non-PTC heater element in thermal contact with a second foraminous heat conductor.

If the operating temperature of the heater element (or heater more generally) is at a lower temperature range (such as from about 20° C. to about 100° C. for example), then a greater amount of the foraminous material in the heat conductor may be preferred in order to achieve the desired heat transfer rate. Using lower temperatures might be preferable from an energy and safety perspective. On the other hand, if the operating temperature range of the heater element (or heater more generally) is at a higher temperature range (for example, from about 100° C. to about 300° C. for PTC heater elements in thermal contact with a heat conductor, or from about 100° C. to about 500° C. for non-PTC heater elements), then less foraminous material may be required to achieve the desired heater transfer rate. Therefore, if a more compact humidifier is desired, then a higher operating temperature may be preferable.

The humidifier may further comprise a water sensor coupled to the heater.

The heater element may be a coil.

The heat conductor may be wrapped cylindrically around the heater element.

The humidifier may be configured to warm up in less than about 1 minute.

The humidifier may be configured to warm up in less than about 30 seconds.

The heater element may be temperature controlled via feedback loop.

In another aspect, the present disclosure includes a humidifier, comprising: a gas flow path, a heater in the gas flow path, the heater comprising: a heater element, and a heat conductor in thermal contact with the heating element, wherein the heater element comprises a PTC material.

The PTC material may be ceramic.

The PTC material may be located within an encapsulation structure.

The encapsulation structure may comprise, electrodes, electrically insulating tape, and an aluminium housing.

The heat conductor may be foraminous.

The foraminous heat conductor may have voids in a solid structure

The foraminous heat conductor may be foam.

The foam may be open cell foam.

The foam may be formed from metal or ceramic.

The heater element may self-regulate the upper limit of its temperature.

The heater may be located within a conduit.

The humidifier may have an overflow drain.

The humidifier may further comprise a fluid delivery apparatus.

The fluid delivery apparatus may comprise a fluid reservoir.

The fluid delivery apparatus may be directly coupled to the heat conductor.

The fluid delivery apparatus may drip fluid onto the heat conductor.

The heater may be in a humidifier tub.

The humidifier may be configured to operate at an operating temperature above about 20° C.

The humidifier may be configured to operate in a temperature range between about 60° C. and about 200° C., preferably between about 120° C. and about 150° C.

The heat conductor may comprise one or more of:
nickel
ceramic
copper
stainless steel.

The humidifier may be configured to operate in a temperature range from about 100° C. to about 300° C.

The humidifier may comprise a PTC heater and/or PTC heater element configured to operate in a temperature range from about 20° C. to about 300° C.

The PTC heater and/or PTC heater element may be configured to operate in a first and/or second configuration:
wherein in the first configuration the PTC heater and/or PTC heater element is configured to operate in a first temperature range from about 20° C. to about 100° C., the PTC heater and/or PTC-heater element in thermal contact with a first foraminous heat conductor.
wherein in the second configuration the PTC heater and/or PTC heater element is configured to operate in a second temperature range from about 100° C. to about 300° C., the PTC heater and/or PTC heater element in thermal contact with a second foraminous heat conductor.

The humidifier may comprise a non-PTC heater and/or non-PTC heater element configured to operate in a temperature range from about 20° C. to about 500° C.

The non-PTC heater and/or non-PTC heater element may be configured to operate in a first and/or second configuration:
wherein in the first configuration the non-PTC heater and/or non-PTC heater element is configured to operate in a first temperature range from about 20° C. to about 100° C., the non-PTC heater and/or non PTC-heater element in thermal contact with a first foraminous heat conductor.
wherein in the second configuration the non-PTC heater and/or non-PTC heater element is configured to operate in a second temperature range from about 100° C. to about 500° C., the non-PTC heater and/or non-PTC heater element in thermal contact with a second foraminous heat conductor.

If the operating temperature of the heater element (or heater more generally) is at a lower temperature range (such as from about 20° C. to about 100° C. for example), then it a greater amount of the foraminous material in the heat conductor may be preferred in order to achieve the desired heat transfer rate. Using lower temperatures might be preferable from an energy and safety perspective. On the other hand, if the operating temperature range of the heater element (or heater more generally) is at a higher temperature range (for example, from about 100° C. to about 300° C. for PTC heater elements in thermal contact with a heat conductor, or from about 100° C. to about 500° C. for non-PTC heater elements), then less foraminous material may be required to achieve the desired heater transfer rate. Therefore, if a more compact humidifier is desired, then a higher operating temperature may be preferable.

The humidifier may be configured to warm up in less than about 1 minute.

The humidifier may be configured to warm up in less than about 30 seconds.

In another aspect, the present disclosure includes a humidifier, comprising:

a gas flow path, a heater in the gas flow path, wherein the heater comprises a PTC material and a foraminous material.

The foraminous material may have voids in a solid structure.

The foraminous material may be foam.

The foam may be open cell foam.

The foam may be formed from metal or ceramic.

The PTC material may be ceramic.

The foraminous material may be metal foam or ceramic foam.

The heater may be located within a conduit.

The humidifier may have an overflow drain.

The humidifier may further comprise a fluid delivery apparatus.

The fluid delivery apparatus may comprise a fluid reservoir.

The fluid delivery apparatus may be directly coupled to the heat conductor.

The fluid delivery apparatus may drip fluid onto the heat conductor.

The heater may be in a humidifier tub.

The humidifier may be configured to operate at an operating temperature above about 20° C.

The humidifier may be configured to operate in a temperature range between about 60° C. and about 200° C., preferably between about 120° C. and about 150° C.

The foraminous material may be one or more of:
nickel
ceramic
copper
stainless steel.

The humidifier may be configured to operate in a temperature range from about 100° C. to about 300° C.

The humidifier may comprise a PTC heater and/or PTC heater element configured to operate in a temperature range from about 20° C. to about 300° C.

The PTC heater and/or PTC heater element may be configured to operate in a first and/or second configuration:
wherein in the first configuration the PTC heater and/or PTC heater element is configured to operate in a first temperature range from about 20° C. to about 100° C., the PTC heater and/or PTC-heater element in thermal contact with a first foraminous heat conductor.
wherein in the second configuration the PTC heater and/or PTC heater element is configured to operate in a second temperature range from about 100° C. to about 300° C., the PTC heater and/or PTC heater element in thermal contact with a second foraminous heat conductor.

The humidifier may comprise a non-PTC heater and/or non-PTC heater element configured to operate in a temperature range from about 20° C. to about 500° C.

The non-PTC heater and/or non-PTC heater element may be configured to operate in a first and/or second configuration:
wherein in the first configuration the non-PTC heater and/or non-PTC heater element is configured to operate in a first temperature range from about 20° C. to about 100° C., the non-PTC heater and/or non PTC-heater element in thermal contact with a first foraminous heat conductor.
wherein in the second configuration the non-PTC heater and/or non-PTC heater element is configured to operate in a second temperature range from about 100° C. to about 500° C., the non-PTC heater and/or non-PTC heater element in thermal contact with a second foraminous heat conductor.

If the operating temperature of the heater element (or heater more generally) is at a lower temperature range (such as from about 20° C. to about 100° C. for example), then a greater amount of the foraminous material in the heat conductor may be preferred in order to achieve the desired heat transfer rate. Using lower temperatures might be preferable from an energy and safety perspective. On the other hand, if the operating temperature range of the heater element (or heater more generally) is at a higher temperature range (for example, from about 100° C. to about 300° C. for PTC heater elements in thermal contact with a heat conductor, or from about 100° C. to about 500° C. for non-PTC heater elements), then less foraminous material may be required to achieve the desired heater transfer rate. Therefore, if a more compact humidifier is desired, then a higher operating temperature may be preferable.

The heater may comprise a heater element, and a heat conductor.

The heater element may comprise the PTC material.

The heater element may self-regulate the upper limit of its temperature.

The heat conductor may comprise the foraminous material.

The foraminous material may have voids in a solid structure.

The foraminous material may be foam.

The foam may be open cell foam.

The foam may be formed from metal or ceramic.

The heat conductor may be for ameliorating the Leidenfrost effect.

The heat conductor may be for increasing the heat transfer rate.

The humidifier may be configured to warm up in less than about 1 minute.

The humidifier may be configured to warm up in less than about 30 seconds.

Described herein is a humidifier for humidifying a flow of gas, comprising: a gas flow path, and a foraminous heater in the gas flow path for heating fluid to humidify gas flowing through the gas flow path.

The foraminous heater may have voids in a solid structure

The foraminous heater may be foam.

The foam may be open cell foam.

The foam may be formed from metal or ceramic.

The humidifier may comprise a fluid delivery apparatus for delivering fluid to the foraminous heater.

The fluid delivery apparatus may comprise fluid reservoir for holding a fluid.

The foraminous heater may comprise a heater element and a foraminous heat conductor.

The heater element and the foraminous heat conductor may be disposed within the tub.

The heater element may comprise a positive temperature coefficient (PTC) material.

The heater element may self-regulate the upper limit of its temperature.

The foraminous heat conductor may have voids in a solid structure.

The foraminous heat conductor may be formed from foam.

The foam may be open cell foam.

The foam may be formed from metal or ceramic.

The foraminous heat conductor may be a metal foam.

The humidifier may be:
a standalone humidifier for connection to a flow of gas
a humidifier for removable connection to a flow source
a humidifier forming part of a flow source.

In another aspect, the present disclosure includes a tub for a humidifier, comprising: a body for humidification, a first heat conductor and a second heat conductor arranged to form a humidification region and/or gas flow path in the body between the first heat conductor and the second heat conductor, and wherein the second heat conductor is foraminous to allow gas to pass to and from the humidification region and/or gas flow path.

The second heat conductor may have voids in a solid structure.

The second heat conductor may be foam.

The foam may be open cell foam.

The foam may be formed from metal or ceramic.

The tub may be adapted to receive an injection of water.

The tub may be adapted to receive the injection of water from a fluid delivery apparatus.

The tub may be adapted to be installed in a humidifier with a heater element to heat the first and/or second heat conductor.

The heating element may comprise a positive temperature coefficient (PTC) material.

The humidifier may be:
a standalone humidifier for connection to a flow of gas
a humidifier for removable connection to a flow source
a humidifier forming part of a flow source.

In another aspect, the present disclosure includes a humidifier for humidifying a gas, comprising: a body for humidification, a first heat conductor and a second heat conductor arranged to form a humidification region and/or gas flow path in the body between the first heat conductor and the second heat conductor, and a heating element for heating the first and/or second heat conductor, wherein the second heat conductor is foraminous to allow gas to pass to and from the humidification region.

The second heat conductor may have voids in a solid structure.

The second heat conductor may be foam.

The foam may be open cell foam.

The foam may be formed from metal or ceramic.

The humidifier may further comprise a fluid delivery apparatus for injecting fluid onto the first and/or second heat conductor.

The first and/or second heat conductor may be adapted to substantially vaporise fluid injected by the fluid delivery apparatus when fluid contacts the first and/or second heat conductor.

The humidifier may be:
a standalone humidifier for connection to a flow of gas
a humidifier for removable connection to a flow source
a humidifier forming part of a flow source.

The second heat conductor may be adapted to vaporise aerosolised fluid when aerosolised fluid contacts the second heat conductor.

The humidifier may further comprise a tub.

The first and second heat conductors may be disposed within the tub.

The first and/or second heat conductor may be foraminous.

The foraminous first and/or second heat conductor may have voids in a solid structure.

The foraminous first and/or second heat conductor may be foam.

The foam may be open cell foam.

The foam may be formed from metal or ceramic.

The first and/or second heat conductor may be a metal foam.

The first heat conductor may be thermally connectable to the heating element.

The heating element may comprise a positive temperature coefficient (PTC) material.

The heating element may self-regulate the upper limit of its temperature.

The humidifier may be:
a standalone humidifier for connection to a flow of gas
a humidifier for removable connection to a flow source
a humidifier forming part of a flow source

Described herein is a medical gases delivery system with a humidifier according to any one or more of the statements or embodiments described herein.

The medical gases delivery system may further comprise a patient interface.

The patient interface may be one or more of:
sealed nasal cannula
non-sealing nasal cannula
nasal mask
facial mask
oro-nasal mask
endotracheal tube
tracheostomy tube
nasal pillow
diffuser
cannula

Also described herein is a medical gases delivery system with a humidifier coupled to or for coupling to a gas source, wherein the gas source is one or more of:
a ventilator
a flow generator
a wall source of gas
a gas bottle
a blender.

Also described herein is a medical gases delivery system with a humidifier coupled to or for coupling to a flow source.

The flow source may provide one or more of:
ventilation
invasive therapy
non-invasive therapy
CPAP therapy
bilevel positive airway pressure therapy
high flow therapy
surgical humidification.

The flow source may comprise a flow generator, insufflator or other gas source.

The medical gases delivery system may further comprise or be used with a gas source, wherein the gas source is one or more of: a ventilator, a flow generator, a wall source of gas, a gas bottle, or a blender, and is coupled to or can be coupled to the flow source and/or humidifier.

Also described herein is a medical gases delivery system with a flow source and humidifier that are integrated to form an integrated flow source.

The humidifier may be a modular component that couples to the flow source to provide the integrated flow source, optionally removably.

The humidifier and the flow source may be formed in a single housing to form an integrated flow source.

Also described herein is a flow source with a humidifier according to any of the previous statements, wherein the humidifier comprises: a gas flow path, and a heater in the gas flow path.

The heater may comprise: a heater element, and a heat conductor in thermal contact with the heating element, wherein the heat conductor is foraminous.

The heater may comprise: a heater element, and a heat conductor in thermal contact with the heating element, wherein the heater element comprises a PTC material.

The heater may comprise a PTC material and a foraminous material.

The foraminous material may have voids in a solid structure.

The foraminous material may be foam.

The foam may have open cell foam.

The foam may be formed from metal or ceramic.

Also described herein is a method of humidifying a flow of gases using a humidifier comprising a heater, wherein the method comprises the steps of: heating the heater, feeding a flow of gases into the humidifier, and feeding fluid to the heater such that fluid vaporises upon contact with the heater.

The method may further comprise the step of pumping fluid from a fluid reservoir to the heater.

The step of feeding fluid to the heater may comprise dripping fluid onto the heater.

The humidifier may comprise a controller configured to perform the method steps.

The heater may comprise a heat conductor, and fluid is dripped onto the heat conductor.

The heat conductor may be foraminous.

The foraminous material may have voids in a solid structure.

The foraminous material may be foam.

The foam may be open cell foam.

The foam may be formed from metal or ceramic.

The humidifier may operate as part of a medical gases delivery system according to any previous statement.

The method may further comprise the step of feeding water onto a heater of a humidifier comprises using: a fluid reservoir, a pump, and a feed-line, wherein the method comprises the steps of: operating the pump to pump fluid from a fluid reservoir through a feed-line, and feeding fluid to the heater.

Fluid fed to the heater may be provided at an adjustable flow rate determined based on desired humidity and/or flow of gas through the humidifier.

The step of feeding fluid to the heater may comprise dripping fluid onto the heater.

The humidifier may comprise a controller configured to perform the method steps.

One or more of the fluid reservoir, pump and feed-line may operate as part of a fluid delivery apparatus.

The humidifier may operate as part of a medical gases delivery system according to any previous statement.

Also described herein is a method of heating a heater for a humidifier according to any of the previous statements, the heater comprising a heater element, and a heat conductor, wherein the method comprises the steps of operating the heater element, and conducting heat to the heat conductor.

The heater element may comprise PTC material.

The heat conductor may be foraminous.

The foraminous material may have voids in a solid structure.

The foraminous material may be foam.

The foam may be open cell foam.

The foam may be formed from metal or ceramic.

The heater element may be operable by a controller, such that the controller is configured to regulate operating temperature of the heater element.

The humidifier may operate as part of a medical gases delivery system according to any previous statement.

In this specification, the terms “foraminous” and “porous” are intended to have the same meaning and these terms are interchangeable. The terms “foraminous” and “porous” refer to a structure with void spaces (which may be referred to as “pores”). The void spaces within a foras thminous/porous material may be randomly distributed or uniformly distributed. A foraminous/porous object refers to an object comprising a foraminous/porous material.

The terms “foam”, “open cell foam”, “closed cell foam”, “metal foam”, and “ceramic foam” are used throughout this specification. These terms are well understood in the art.

In this specification the term “flow source” refers to an apparatus that generates a flow of gas, e.g. by rotation of an impeller or similar via a motor or similar. By way of non-limiting examples, the flow source could be or comprise one or more of: a flow generator (as used in respiratory systems), a flow controller (such as an insufflator (used in surgical humidification systems) for example), other flow source, or a gas source (which might be the same as or different to and/or combined with or separate to a gas source (as described herein)).

In this specification, “high flow” means, without limitation, any gas flow with a flow rate that is higher than usual/normal, such as higher than the normal inspiration flow rate of a healthy patient. It can be provided by a non-sealing respiratory system with substantial leak happening at the entrance of the patient's airways due to non-sealing patient interface prongs. It is also provided with humidification to improve patient comfort, compliance and safety. Alternatively or additionally, it can be higher than some other threshold flow rate that is relevant to the context—for example, where providing a gas flow to a patient at a flow rate to meet inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult child) and the like. Those skilled in the art know from context what constitutes “high flow”. It is a magnitude of flow rate that is over and above a flow rate that might otherwise be provided.

But, without limitation, some indicative values of high flow can be as follows.
In some configurations, delivery of gases to a patient at a flow rate of greater than or equal to about 5 or 10 litres per minute (5 or 10 LPM or L/min).
In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a therapy. Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The therapeutic flow can be set to 0.4-8 L/min/kg with a minimum of about 0.5 L/min and a maximum of about 25 L/min. For patients under 2 kg maximum flow is set to 8 L/min.

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### BRIEF DESCRIPTION OF THE FIGURES

FIG. 1 is a side view of a conventional humidifier.

FIG. 2A is one general embodiment of the medical gases delivery system.

FIG. 2B is another general embodiment of the medical gases delivery system.

FIG. 2C is another general embodiment of the medical gases delivery system.

FIG. 2D is another general embodiment of the medical gases delivery system.

FIG. 2E is another general embodiment of the medical gases delivery system.

FIG. 3A is one general embodiment of the humidifier.

FIG. 3B is another general embodiment of the humidifier.

FIG. 3C is another general embodiment of the humidifier.

FIG. 3D is another general embodiment of the humidifier.

FIG. 4 demonstrates why pathogens can be carried by aerosolised water droplets, but cannot be carried by water vapour.

FIG. 5A demonstrates how the Leidenfrost effect delays vaporisation of liquid water.

FIG. 5B demonstrates the use of a foraminous heat conductor to weaken the Leidenfrost effect to assist with vaporisation of water.

FIG. 6 is one embodiment of the humidifier.

FIG. 7 is one embodiment of a PTC heater element incorporated as part of an encapsulation structure.

FIG. 8 is a line graph demonstrating how electrical resistance of a PTC material exponentially increases with temperature.

FIG. 9 is another embodiment of the humidifier.

FIG. 10 is another embodiment of the humidifier.

FIG. 11 is another embodiment of the humidifier.

FIG. 12 demonstrates use of a foraminous aerosol filter used as part of a tub.

FIG. 13 is an alternative embodiment of the fluid delivery apparatus.

FIG. 14 shows a surgical humidification system embodiment of the medical gases delivery system.

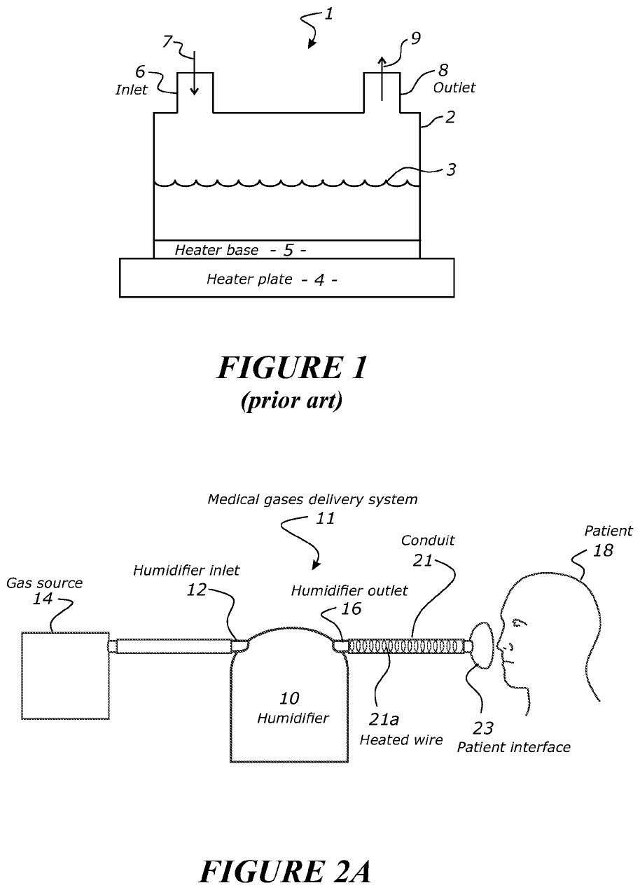

### DETAILED DESCRIPTION

### 1. General Embodiment

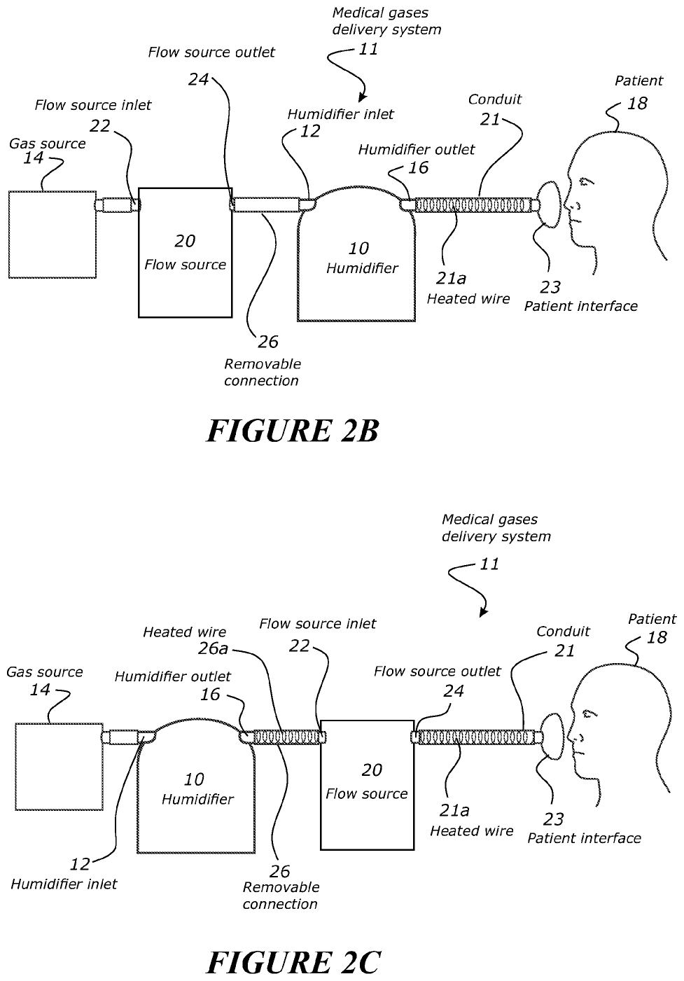

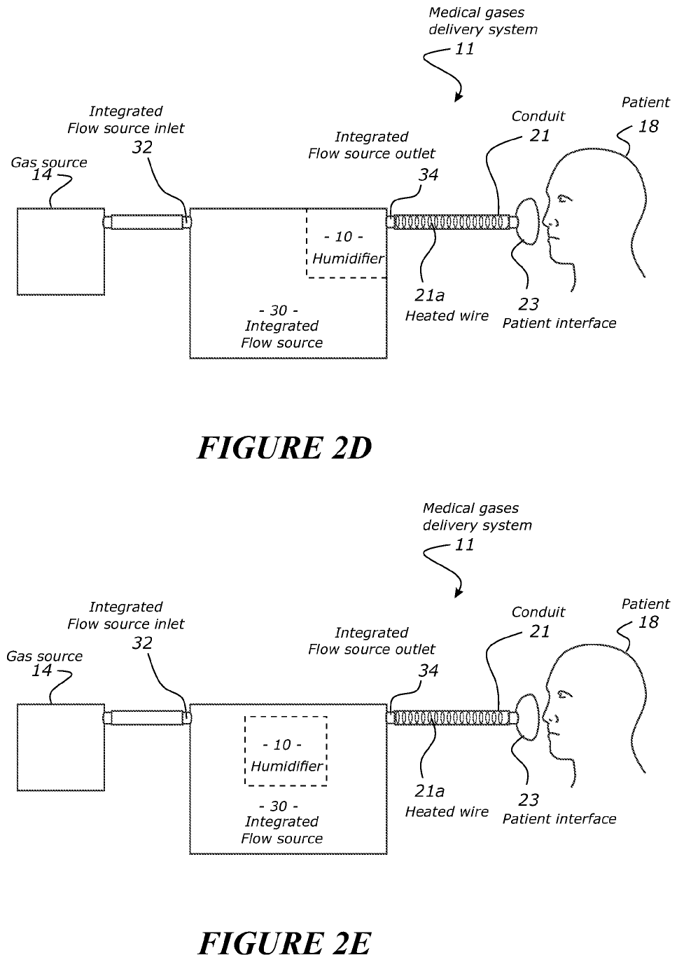

Embodiments of a medical gases delivery system 11 and the humidifier 10 will first be discussed at a general level with reference to FIGS. 2A-2E, 3A-3D, 4, and 5A-5B.

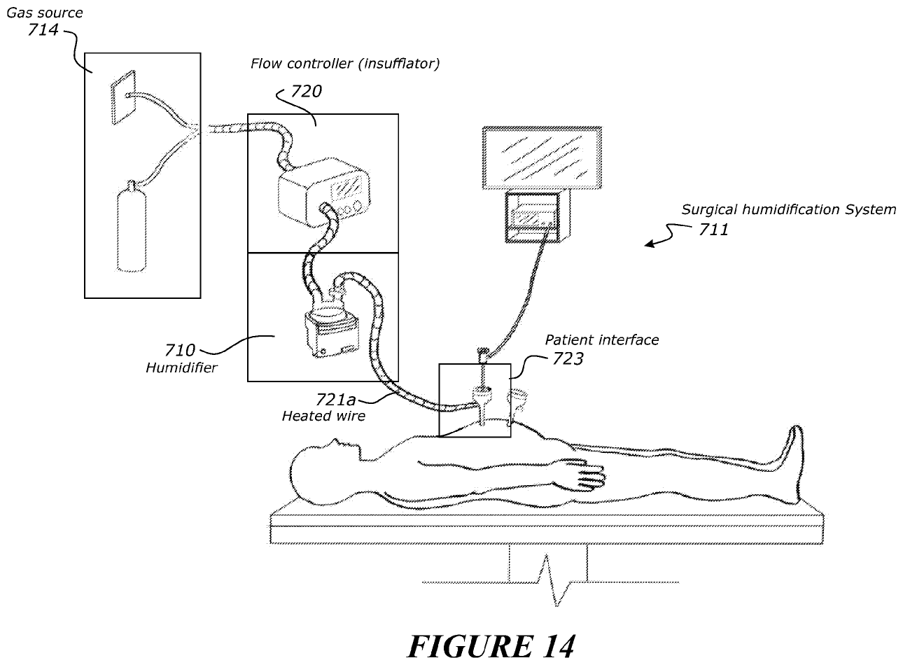

FIGS. 2A-2E, and 14 illustrate embodiments of the medical gases delivery system 11, each embodiment having a humidifier 10 and providing humidified gases to a patient e.g. though a patient interface to the mouth and/or nose; or to another part of the body, such as through an interface into a surgical opening for a surgical procedure. In the case of providing gases through a patient interface to the mouth and/or nose, the medical gases system could be termed a “respiratory system”. In the case or providing gases into a surgical opening during a medical procedure, the medical gases system could be termed a “surgical humidification system”. For context, the humidifier 10 refers to a component or a part of a component that carries out the function of humidifying a flow of gas for delivery to a patient. Such humidification can be used to condition/control the humidity and temperature of a gas that the patient inspires. Unless context dictates otherwise, reference to “humidity” in this specification refers to absolute humidity and/or relative humidity. The embodiments shown in FIGS. 2A-2E, and 14 are exemplary and not necessarily exhaustive. They are also functional/diagrammatic in nature, so actual configurations might vary or look different. The connections described are exemplary and functional in nature and may take any form suitable to achieve connections.

FIG. 2A illustrates a standalone humidifier 10, comprising a humidifier inlet 12, and a humidifier outlet 16. The humidifier inlet is connectable to a gas source 14, which may be one or more of a ventilator, flow generator (which may be turbine or blower based), a wall source of gas, a gas bottle, a blender, or the like. The gas source may form part of the system 11, or could be considered separate. The humidifier 10 is connectable to a patient 18 receiving the flow of humidified gas via a heated breathing circuit, configured to prevent/reduce condensation of the humidified flow of gases downstream from the humidifier 10. The heated breathing circuit comprises the humidifier outlet 16 and/or a conduit 21 and/or patient interface 23. The patient interface 23 can be a sealed patient interface, or a non-sealing patient interface. Some examples of a patient interface could include but are not limited to: a sealed nasal cannula, non-sealing nasal cannula, nasal mask, facial mask, oro-nasal mask, endotracheal tube, tracheostomy tube, nasal pillow, diffuser, cannula, and the like. The patient interface 23 may be directed to any part of the patient's body. The conduit 21 preferably has a heated wire 21a that heats the humidified flow of gases to prevent/reduce condensation of the humidified flow of gases.

FIG. 2B illustrates a humidifier 10 connected to a standalone flow source 20 (e.g. a blower for respiratory therapy/systems or insufflator for surgical humidification), comprising an inlet 22 and outlet 24. Both the humidifier 10 and the standalone flow source 20 are separate devices in this instance. The flow source 20 can be used for any type of medical gases delivery system, comprising but not limited to: ventilation, invasive therapy, non-invasive therapy, CPAP therapy, bilevel positive airway pressure high flow therapy, surgical humidification, and/or any other respiratory related treatment, and/or other surgical related treatment, and the like. The flow source could be or comprise one or more of: a flow generator, a flow controller (such as an insufflator for example), other flow source, or a gas source (which might be the same as or different to and/or combined with or separate to the gas source 14). The inlet 22 is connectable to a gas source 14, which may be a ventilator, flow generator (which may be turbine or blower based), a wall source of gas, a gas bottle, a blender, or the like. The outlet 24 is connectable to the humidifier inlet 12. This might be by way of a connection 26, which may be separate and/or removable and/or may form part of the outlet. The other end of the connection 26 is connectable to the humidifier inlet 12, which may be separate and/or removable and/or may form part of the inlet. The humidifier 10 is connectable to a patient 18 receiving the flow of humidified gas via a heated breathing circuit, configured to prevent condensation of the humidified flow of gases downstream from the humidifier 10. The heated breathing circuit comprises the humidifier outlet 16 and/or a conduit 21 and/or patient interface 23. The conduit 21 preferably has a heated wire 21a that heats the humidified flow of gases to prevent condensation of the humidified flow of gases.

FIG. 2C illustrates another example, in which the standalone humidifier 10 and flow source 20 devices are positioned in reverse order (from the example of FIG. 2B) such that the humidifier 10 is positioned pneumatically upstream from the flow source, which is what is shown in FIG. 2C. The humidifier inlet 12 is connectable to a gas source 14, which may be a ventilator, flow generator (which may be turbine or blower based), a wall source of gas, a gas bottle, a blender, or the like. The humidifier outlet 16 is connectable to the inlet 22 of the flow source 20. This might be by way of a connection 26, which may be separate and/or removable and/or may form part of the outlet. The other end of the connection 26 is connectable to the inlet 22 of the flow source 20, which may be separate and/or removable and/or may form part of the inlet. The flow source could be or comprise one or more of: a flow generator, a flow controller (such as an insufflator for example), other flow source, or a gas source (which in terms of type might be the same as or different to the gas source 14). The removable connection 26 preferably has a heated wire 26a that heats the flow of gases to prevent condensation anywhere along the removable connection 26. The outlet 24 of the flow source 20 is connectable to a patient 18 receiving the flow of humidified gas via a conduit 21 and/or patient interface 23. The humidifier 10 is therefore connectable to the patient 18 receiving the flow of humidified gas via a heated breathing circuit, configured to prevent condensation of the humidified flow of gases downstream from the humidifier 10. The heated breathing circuit comprises the humidifier outlet 16 and/or the removable connection 26 and/or the flow source 20 and/or the inlet/outlet of the flow source 22, 24 and/or the conduit 21 and/or the patient interface 23. The conduit 21 and removable connection 26 preferably have a respective heated wire 21a, 26a that heats the humidified flow of gases to prevent condensation of the humidified flow of gases.

FIGS. 2D and 2E illustrate a humidifier 10 and flow source integrated into a single integrated flow source housing. The humidifier 10 may be integrated into the integrated flow source 30, in which the extent of integration lies at any point along a spectrum extending from a modular add-on component that fits onto (optionally removably) the rest of the integrated flow source 30 (as shown in FIG. 2D for example), to the other end of the spectrum where the functions of the humidifier 10 are fully integrated into the flow source 30, (as shown in FIG. 2E for example). The integrated flow source could comprise one or more of: a flow generator, a flow controller (such as an insufflator for example), other flow source, or a gas source (which might be the same as or different to and/or combined with or separate to the gas source 14). In the examples demonstrated in FIGS. 2D and 2E, the inlet 32 of the integrated flow source 30 is connectable to a gas source 14, which may be a ventilator, flow generator (which may be turbine or blower based), a wall source of gas, a gas bottle, a blender, or the like. In the examples demonstrated in FIGS. 2D and 2E, the integrated flow source 30 is connectable to a patient 18 receiving the flow of humidified gas via a heated breathing circuit, configured to prevent condensation of the humidified flow of gases downstream from the humidifier 10. The heated breathing circuit comprises any portion of the integrated flow source 30 downstream of the humidifier 10 and/or the integrated flow source outlet 34 and/or a conduit 21 and/or patient interface 23. The conduit 21 preferably has a heated wire 21a that heats the humidified flow of gases to prevent condensation of the humidified flow of gases.

FIG. 14 illustrates a surgical humidification system 711 as an exemplary embodiment of the medical gases delivery system 11. The surgical humidification system 711 shown has a gas source 714 (embodiment of gas source 14), a flow controller (e.g. insufflator) 720 (an embodiment of flow source 20), humidifier 710 (embodiment of humidifier 10), heated wire 721a (embodiment of heater wire 21a), and patient interface 723 (embodiment of patient interface 23). The patient interface 723 shown in FIG. 14 is directed to the patient's body.

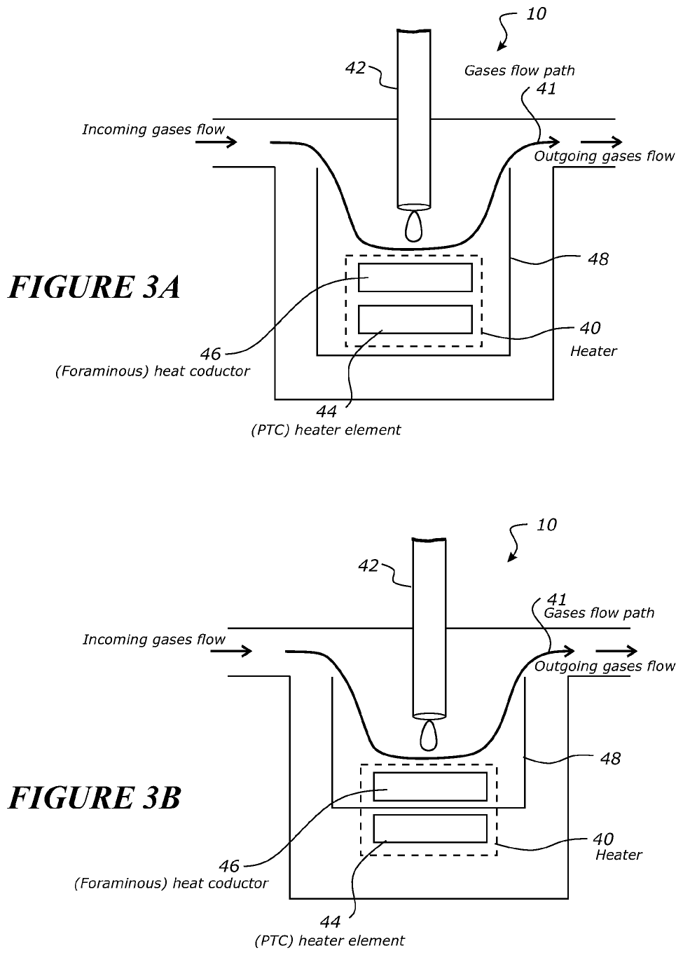

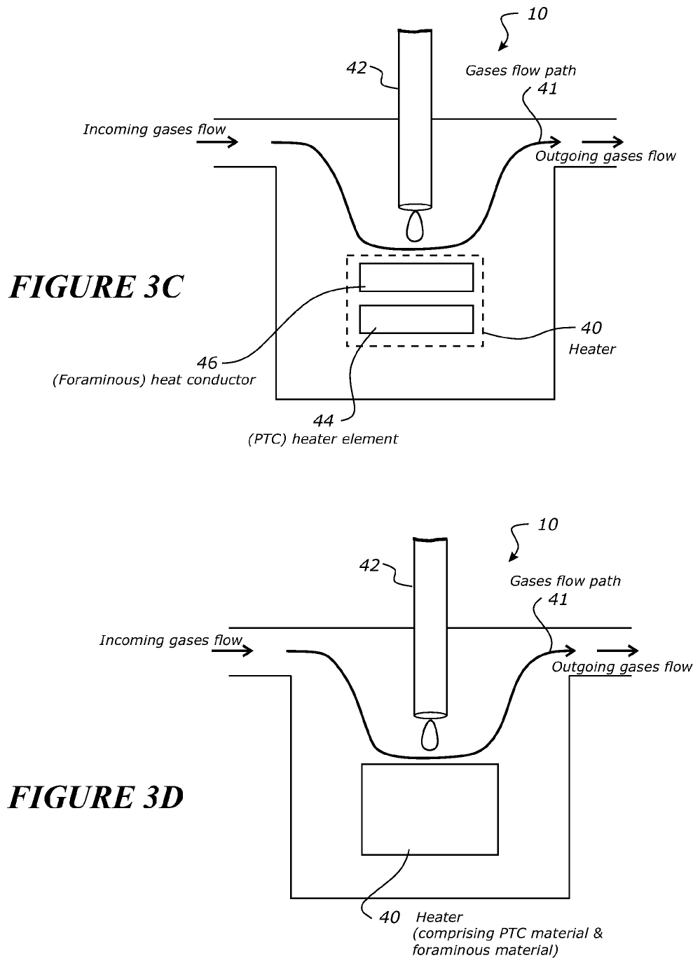

FIGS. 3A-3D illustrate embodiments of the humidifier 10 at a general level.

The humidifier 10 has a heater 40 (located in the gas flow path 41) and a fluid injector 42 (a term that is interchangeable with the terms “fluid feeder” or “fluid delivery apparatus”). In use, the fluid feeder 42 feeds fluid to the heater 40, and the heater 40 (including body and/or surfaces) is heated to vaporise the fluid provided by the fluid feeder 42. The fluid may be provided at any suitable rate or form (droplets, steady stream, injected from within the heater etc.) from any suitable location (above the heater 40, from within the heater, etc.). When the fluid either contacts and/or is close enough to the heater 40, the heater vaporises the fluid. The vaporised fluid mixes with the incoming flow of gas to humidify the gas for delivery to the patient 18. It is preferable that liquid water is used as the fluid, and for the rest of the specification, it is assumed that the fluid feeder 42 is for feeding small quantities of liquid water onto the heater 40, although a skilled person would realise that other fluids may be suitable, for instance a medicament.

The heater 40 may comprise a heater element 44, and one or more heat conductors 46 (which can also be termed “conductive heater”). The heater element 44 is a heat source, in which thermal energy is converted from another energy type. For example, the heater element 44 is configured to convert electrical energy into thermal energy to generate a heat source. The heater element 44 is configured to achieve a rapid start up shortly after the humidifier 10 is switched on. The heater element 44 may comprise a positive temperature coefficient (PTC) material (can be termed “PTC heater element”, and termed “PTC heater” more generally), such as ceramic for example, although this is not essential in some embodiments (i.e. these embodiments have non-PTC heater and/or non-PTC heater element). The properties of a PTC material will be discussed later. The heat conductor 46 is thermally connected to the heater element 44, and therefore conducts heat away from the heater element 44. The heater element 44 heats up the surface of the heat conductor 46 to a high enough temperature to rapidly vaporise a controlled quantity of water. The heat conductor 46 may comprise a foraminous/porous material (i.e. foraminous heat conductor) capable of conducting thermal energy, such as a metal foam for example. It is appreciated that the heater 40 may also be described as a heater that comprises a PTC material, and a foraminous/porous material. The foraminous/porous material can be any material that can be fabricated to be foraminous/porous (i.e. have a foraminous/porous structure) via a manufacturing method/process. In some embodiments, the foraminous/porous material is a metal or ceramic made (via a manufacturing method/process) into a foam structure. That is, the foam is formed from metal or ceramic. The foam structure can optionally be formed from voids in a solid structure. The foam structure can optionally be open cell foam. It is desirable that the (foraminous/porous) material selected for the heat conductor 46 does not oxidise well, or if it does, it oxidises to a stable and tenacious oxide, “sealing” the underlying (foraminous/porous) material from further oxidation. This is to address longer term usage and concerns about metal salts and the effect of those on the metal. Water constituents may affect the longevity of the heat conductor material. It is acceptable having small quantities of salt deposits in the water, as long as they do not deposit in sufficient quantities to block the pores in the (foraminous/porous) material. It is also desirable when selecting a (foraminous/porous) material for the heat conductor that the pore size of the material is sufficient to enable the foraminous/porous material to get wet.

In some embodiments, such as those shown in FIGS. 3A and 3B, the humidifier 10 can comprise a vaporisation tub (“humidifier chamber”) 48 for where vaporisation takes place. The purpose of the (vaporisation) tub 48 is to facilitate the vaporisation of small quantities of water. The tub 48 comprises an internal heat conductor 46. The tub 48 can be a detachable component such that it is insertable into the humidifier 10, and removable from the humidifier 10. Alternatively, the tub 48 can be built-in component of the humidifier 10. In the example shown in FIG. 3A, the tub 48 comprises a heater element 44 and heat conductor 46, such that the heater 40 is fully contained within the tub 48. In the example shown in FIG. 3B, the tub comprises a heat conductor but not necessarily a heater element 44. In this example, the heater 40 is formed when the tub 48 is inserted into the humidifier 10 so that the heater element 44 and the heat conductor 46 are in thermal contact with each other. In some embodiments, the humidifier 10 does not comprise a tub 48, see FIG. 3C. The heater 40 forms part of the humidifier. The embodiments of FIGS. 3A-3C may have a heater 40 that comprises a PTC heater element 44 and/or foraminous heat conductor 46.

It is envisioned that the humidifier 10 may be a) a standalone “box” unit, which can be used on its own or coupled to a flow source (such as a blower), b) incorporated as part of an integrated “box” unit comprising a humidifier and flow source, or c) incorporated as part of any other component of the medical gases delivery system 11 (for example, the humidifier 10 may be incorporated as part of a removable connection 26, in the patient conduit 21, or the like).

The humidifiers 10 as described above may be removable or portable. They may be battery powered, mains powered, or both. The humidifier 10, or any component of the humidifier 10 may be battery powered 350, 450 (AC, DC, or both).

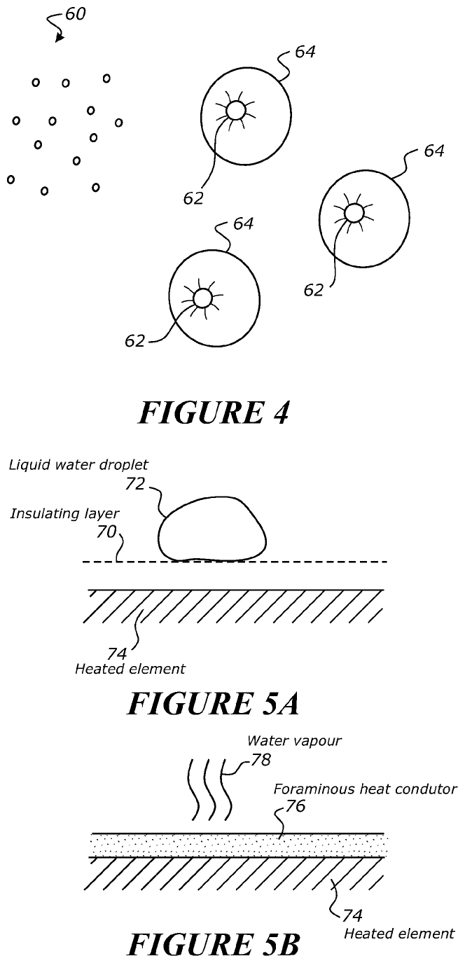

Embodiments of the humidifier 10 described above, which has a heater 40 comprising a heater element 44 and at least one heat conductor 46, may provide one or more of these advantages:
First, for health and safety reasons, it is desirable to heat the heater 40 such that the heater 40 heats the water to a temperature at or greater than the desired dew point temperature (this could be any suitable temperature, for example, between about 29° C. and about 37° C. Typical examples are about 29° C., about 31° C., or about 37° C. for example, depending on the therapy being administered (such as ventilation, invasive, non-invasive, bilevel positive airway pressure, high flow therapy, surgical humidification, and/or any other respiratory related treatment, and/or any other surgical related treatment, and the like)). The humidifier 10 can be operated to heat water to the desired operating temperatures as mentioned.
Second, the foraminous heater 40 vaporises the water such that only water vapour is present, and aerosols are absent. Referring to FIG. 4, vaporisation is desired because water vapour 60 is too small to transport pathogens 62. On the other hand, aerosols 64 (caused by aerosolization (which for example could include high flow or shear rates across any liquid water/air interface, spitting caused by high temperatures, the Leidenfrost effect, condensation, and the like)) are much bigger in size than water vapour 60, and are big enough to transport pathogens 62, thus posing a contamination issue. It is therefore desirable that the heater 40 is heated to a temperature about, at, or above the desired dew point temperature so that water provided by the fluid feeder 42 vaporises into water vapour upon contacting the hot surface of the heater 40. To reduce instances of hospital acquired infections, it is advantageous to reduce and/or substantially prevent aerosols (capable of carrying pathogens) from being generated or formed.
Third, when the user starts up the humidifier. Unlike conventional humidifiers, which requires heating of a body of water in a humidifier chamber, the heater element does not heat up a substantial body of water. Instead, the heater element only needs to heat up the heat conductor, as opposed to heating a chamber full of water (which has a much higher heat capacity than the heat conductor). This leads to a faster warm-up time. The reduction in the heat capacity of the system (by reducing heat capacity of foam, heater (as the heater size is reduced), and water (as the amount of water is reduced)), leads to a faster warm-up time.
Fourth, with reference to FIGS. 5A and 5B by way of example, the foraminous heat conductor can be used to ameliorate the Leidenfrost effect, which is a physical phenomenon which prevents rapid vaporisation of liquid, because the liquid is close to a mass that is significantly hotter than the liquid's boiling point. Under such circumstances, an insulating vapour layer 70 is formed above the heated surface that prevents liquid 72 from contacting a heated element 74 and thus the liquid droplet 72 hovers above the heated element 74 instead. In contrast, the foraminous heat conductor 76 weakens the insulating layer causing the Leidenfrost effect, thereby ameliorating the Leidenfrost effect, and reducing the vaporisation delay produced as a result of the Leidenfrost effect. In the humidifiers described, the foraminous heat conductor can be used to ameliorate the Leidenfrost effect when the heater element (or heater more generally) operates at (about) 100° C. or above. If there was no foraminous heat conductor 76, and water is instead dropped onto the surface of the heater element 74 (the temperature heater element surface can be about, at or greater than the Leidenfrost temperature (which is dependent on the fluid and heat conductor material—for example the Leidenfrost temperature could be as low as 127° C. or even lower), then the insulating layer 70 creating the Leidenfrost effect would significantly delay the water from vaporising. In addition, the liquid water droplets 72 that form above the insulating layer 70 “split”, spatter and can generate aerosols 64. Aerosols 64, unlike water vapor 60, can carry pathogens 62, therefore creating an issue with contamination, as shown in FIG. 4.
Fifth, the humidifier produces a reduced output response time for two reasons.
   First, the porosity of the foraminous heat conductor 76 helps provide a large interface area between the heater and the liquid water, which increases maximum vaporisation rate. This is made possible, because the foraminous heat conductor 76 provides a capillary action that wicks the water so that discrete “blobs” of water are “spread out” by capillary action to form a liquid film “spread” throughout the foraminous heat conductor 76. The capillary action also increases the liquid air interface area, which also increases the maximum vaporisation rate.
   Second, the minimum required volume of liquid water is provided to the heater, restricting the amount of water being heated at any given time. This reduces the water heat capacity meaning a faster response time.
Sixth, the humidifier can be thermally efficient. The total volume of the humidifier, (and the total surface area of the humidifier exposed to ambient) is relatively compact. The volume is expected to be lower than traditional humidifiers, this means a correspondingly larger reduction in humidifier external surface area exposed to ambient temperatures, which will reduce thermal loss. This is possible because the thermal energy loss to ambient is minimised by the relatively compact surface area of the heater. Further, the heater is fully encapsulated by the flow of gases, meaning the thermal loss from the heater to ambient is reduced even further.
Seventh, for PTC heater elements 44, to improve (e.g. maximise) heat flux out of the heater, and therefore improve (e.g. maximise) heat transfer rates into the fluid, both sides of the heater element 44 can and should be constructed to be in good thermal contact with the heat conductor 46.
Eighth, the heater 40 allows for a different type of humidification control. Due to the high operating temperatures of the heater 40, it can be reasonably assumed that all water fed by the fluid feeder 42 is being vaporised. This means humidification can be controlled by how much water is being supplied by the fluid feeder 42. This is a different control mechanism to conventional humidifiers, which instead control humidification by controlling the temperature of the water. Further, since all liquid water provided by the fluid feeder 42 can be vaporised by the heater, the heated breathing circuit (comprising any portion of the medical gases delivery system 11 downstream from, and including the humidifier 10) has an added advantage of being able to be orientated in any position without consideration to liquid water spillage, since liquid water is provided into the humidifier 10 from an external reservoir.
Ninth, use of the PTC material helps to self-regulate the operating temperature of the heater element 44 without having to use a controller to control operation of the heater element 44. The electrical resistance of the PTC material exponentially increases as the temperature of the PTC material increases. This limits the temperature of the PTC material and prevents thermal runaway.
Tenth, unlike conventional humidifiers, the humidifier 10 can operate independently of the humidifier inlet temperature due to the high operation temperature of the humidifier 10, which still maintains a heated and humidified output within temperature and enthalpy safety limits.
Eleventh, the enthalpy of the humidifier 10 is lower due to the lower heat capacity of the humidifier 10. This means there is a lower chance of temperature overshoot, making the humidifier 10 safer for use.

Combinations of the advantages described above can result in a “fast acting” humidifier 110 that can be capable of providing a “quick response”—for example, in one or more of the following ways:
The humidifier 10 can provide a maximum humidification and/or heating output relatively quickly (within a few to 30 seconds, or under a minute) from starting up.
The humidifier 10 can cool down relatively quickly (within a few to 30 seconds, or under a minute) once the humidifier 10 is powered off. Such a property may be desirable for some medical therapies, such as surgical humidification applications.
The humidifier 10 can relatively quickly (within a few to 30 seconds, or under a minute) ramp up the relative and/or absolute humidity of gas delivered to the patient.
The humidifier 10 can relatively quickly (within a few to 30 seconds, or under a minute) ramp up the temperature of gas delivered to the patient.
The humidifier 10 can relatively quickly (within a few to 30 seconds, or under a minute) reduce (i.e. “drop”) the relative and/or absolute humidity of gas delivered to the patient. This could be useful when adding further substances to the gas flow (such as nebulized, powdered, or metered medicament).
The humidifier 10 can relatively quickly (within a few to 30 seconds, or under a minute) reduce (i.e. “drop”) the temperature of gas delivered to the patient.

Combinations of the advantages described above might also facilitate a construction which results in a small/portable humidifier.

Referring to FIG. 3D, it is appreciated that the heater 40 may alternatively comprise a single component in which the functions of the heater element 44 and heat conductor 46 are integrated into a “single-piece” heater, which could comprise a PTC material and a foraminous material. Such heater may be termed a PTC heater as it comprises PTC material. The heater 40 may or may not be (wholly or partially) in a tub 48. Such an embodiment may have one or more of the advantages listed above.

A skilled person will appreciate that any of the embodiments of humidifier 10 can be used in any situation where humidification is desired, including specific embodiments of humidifier 10 which will be described later.

Discussion now turns to specific embodiments of the humidifier 10.

### 2. First Embodiment

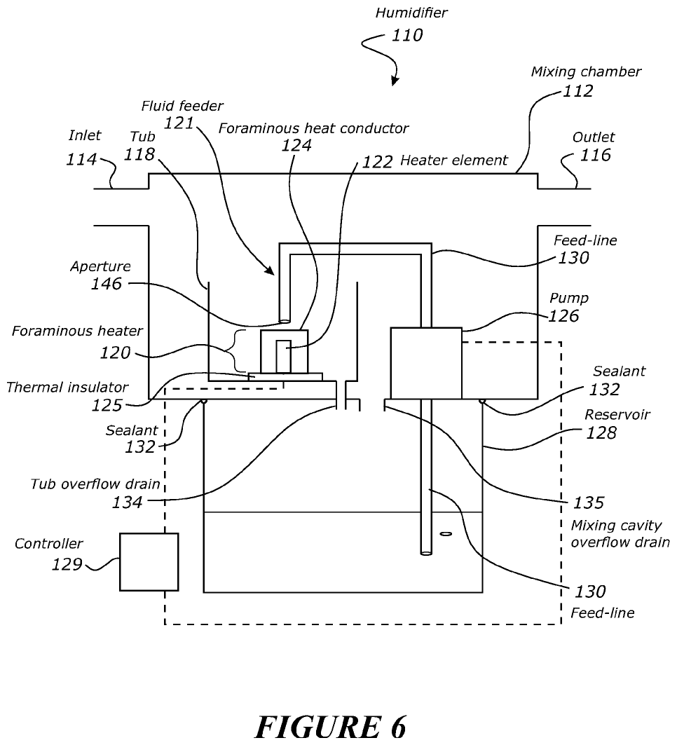

FIG. 6 shows a first embodiment of a humidifier 110. This loosely corresponds to the general embodiment shown in FIG. 3A, and is one possible embodiment of such an arrangement. The humidifier 110 includes a mixing cavity 112 (a term that is interchangeable with “mixing chamber”) for mixing vaporised water with an incoming flow of gas, resulting in humidification of gas. The flow of gas enters the humidifier 110 through the humidifier inlet 114, and humidified gas flow exits the humidifier 110 through the humidifier outlet 116. The mixing chamber 112 comprises a tub 118 containing a foraminous heater 120, and a fluid feeder 121 for dripping water onto foraminous heater 120. The tub 118 is used for vaporising water, and in some embodiments, the tub 118 is uncovered, as shown in FIG. 6.

The foraminous heater 120 comprises a heater element 122, and a foraminous heat conductor 124. The foraminous heat conductor 124 encapsulates the heater element 122. This is done by wrapping the foraminous heat conductor over the outer surface of the heater element 122, thus creating thermal contact between the heater element 122 and the foraminous heat conductor 124. The heater element 122 and foraminous heat conductor 124 are supported by a thermal insulator 125 that is located at the base of the tub 118.

The fluid feeder 121 is used to drip water onto the surface of the foraminous heat conductor 124 in this embodiment. The fluid feeder 121 comprises at least the feed-line 130, but may also additionally comprise a water pump 126 and/or water reservoir 128. The water pump 126 draws water up from the water reservoir 128 along feed-line 130. The water pump 126 then pumps water out along the feed-line 130, through the fluid feeder 121. As a result, fluid is dripped onto the foraminous heat conductor 124. The water reservoir 128 is used for storing a body of water to be used for humidifying the flow of gas. The water reservoir 128 may be sealed off with the use of a sealant 132. The tub 118 may have a tub overflow drain 134, and a mixing cavity overflow drain 135, such that any excess water not vaporised by the foraminous heat conductor 124, or excess water formed by condensation, is drained from the tub 118, and is returned back to the water reservoir 128 for later use.

The humidifier 110 may also have a controller 129 for controlling the operation of the humidifier 110. The controller 129 can refer to one or more controllers configured to operate any functions described in this embodiment herein.

Discussion now turns to the features described in this embodiment in more detail.

2.1 Heater Element

The heater element 122 provides a heat source for the foraminous heat conductor 124 to vaporise water for humidification. Preferably the heater element 122 acts as a heat source by converting electrical energy into thermal energy.

In the embodiment shown in FIG. 6, the heater element 122 is mounted vertically, which increases the utilisation of power, as the heater flux in the heater element 122 is evenly distributed on both sides. That is, the heater element 122 extends vertically from the base of the tub 118, or extends vertically from the thermal insulator base 125 that the heater element 122 is mounted to within the tub 118, with the foraminous heat conductor 124 on either side of the heater element 122. Another way of increasing the utilisation of power provided by the heating element 122 is to design the heater element 122 and the foraminous heat conductor 124 such that water can spread to the underside of the heater element 122 and to the underside of the foraminous heat conductor 124.

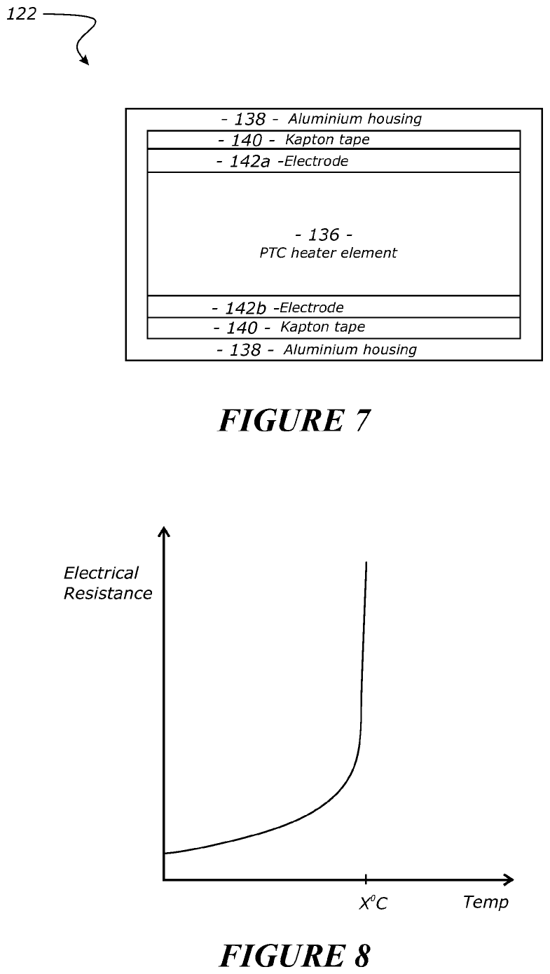

The heater element 122 is preferably a heating element comprising positive temperature coefficient (PTC) material 136 (a term interchangeable with the term “PTC heater element”). PTC materials are produced from ceramics or polymers that are doped. Preferably, the heater element 122 is the embodiment as illustrated in FIG. 7. FIG. 7 shows a heater element 122 configured as a “sandwich” or encapsulation structure, in which the PTC heater element 136 is encapsulated in a “sandwich” comprising, preferably in this order: an aluminium housing 138, Kapton tape 140 (for electrical insulation), electrode 142a, PTC heater element 136, electrode 142b, Kapton tape 140, and the aluminium housing 138. The heater element 122 may refer to the PTC heater element 136 itself, or any combination of additional types of material used in conjunction with the PTC heater element 136. In some embodiments, the PTC heater element 136 may also comprise ceramic material.

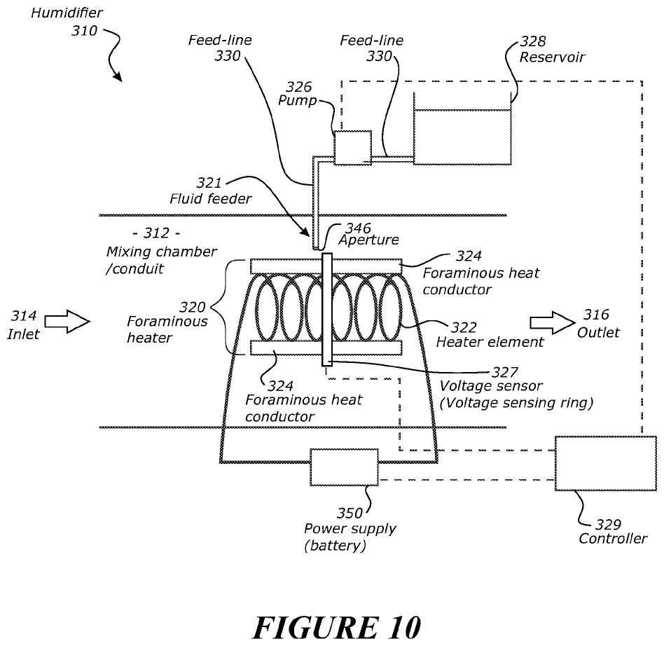

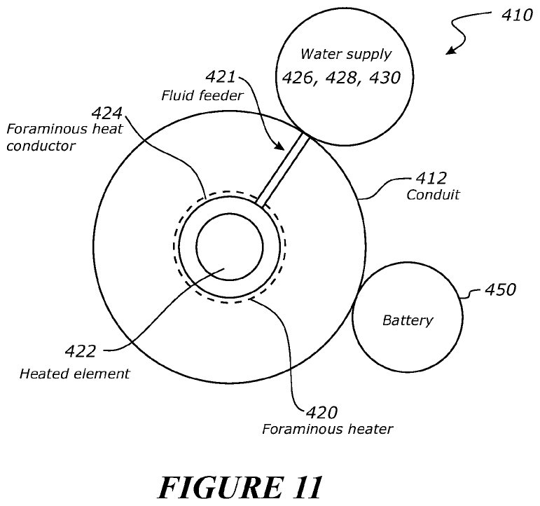

Selection of a PTC material as the material for inclusion in the heater element 136 assists with temperature control of the heater element 136. A PTC material is defined as a material that can self-regulate its temperature. This happens because a temperature increase in the PTC material (when current is passed through the PTC material for heating) causes an increase in electrical resistance, preventing thermal runaway. PTC material used in the heater element 136 should preferably exhibit a highly non-linear/exponential thermal response, so that above a composition-dependent threshold temperature, the resistance of the PTC material in the heater element 136 increases rapidly/exponentially, causing the PTC material in the heater element 136 to act as its own temperature controller. The graph shown in FIG. 8 demonstrates an example of a possible electrical resistance versus temperature profile for a PTC material. In this example shown in FIG. 8, the desired temperature limit is X° C., and the electrical resistance increases rapidly as temperature approaches the limit of X° C. Preferably at this point, the current should drop to about 1% of its initial value. The PTC material inherently prevents thermal runaway, as opposed to requiring control. The thermal property of PTC heater element 136 as described allows measurement of temperature of the PTC heater element 136 without needing to use a temperature sensor for sensing temperature. The current supply to the PTC heater element 136 can be switched off momentarily, so that the electrical resistance of the PTC heater element 136 can be measured. The temperature of the PTC heater element 136 can be inferred if the electrical resistance of the PTC heater element 136 is known, and if the electrical resistance-temperature profile of the PTC material is known. If the electrical resistance is known, the operating temperature can be inferred by querying a look up table, or by calculating an equation that characterises a mathematical relationship between the known electrical resistance and the corresponding temperature of the PTC material used in the heater element 136. Knowledge of the heater element 136 temperature allows for detection of excess water feed from the fluid feeder 121. Vaporisation of the water dripped onto the heater element 136 lowers the operating temperature of the heater element 136 from its expected temperature at the provided current which indicates liquid water is present (and the system has been over-fed). Alternatively, the system could compare the operating power of the PTC heater element to the amount of power expected to be needed to vaporise the amount of water being fed to the system. If the operating power is lower than expected, this indicates that water is accumulating and the system has been over-fed. Based on the description above, it should be apparent to a skilled person that any humidifier embodiments with a PTC heater element will have inherent temperature and power control of the heater element. However, a skilled person would also recognise that other control mechanisms (of which controller 129 can be a component of for example) may optionally be implemented in addition to the inherent temperature and power control provided by the PTC material of the PTC heater element. For example, there may optionally be feedback loops that monitor and/or control electrical resistance (for example, this could be a voltage sensing ring which is later discussed with reference to FIG. 10) or humidity if the appropriate sensor/s and circuitry is provided. Other examples of feedback loops will also be discussed in later humidifier embodiments.

Alternatively, a heater element that does not comprise PTC material (i.e. a non-PTC heater element with a linear thermal response) may be used instead of a PTC heater element. If a non-PTC heater element is used, a method of temperature control (e.g. using the controller) can be implemented to limit temperature and ensure safe operating temperatures on the heater element 136. In a non-PTC heater element, the heater element may have an electrical resistance that does not increase exponentially with an increase in operating temperature. Consequently, a feedback loop is used for temperature control of the heater element, and for maintaining safe operating temperature limits. This also means sensors are used to monitor the temperature of the non-PTC heater element, whether sensing directly or indirectly. It should be apparent to a skilled person that they can implement a feedback loop (including sensor/s) when implementing any (other) humidifier embodiment with a non-PTC heater element.

The controller 129 (i.e. a voltage controller) can be configured to control the voltage and/or current supplied to the heater element 122 to convert electrical energy into thermal energy. If the heater element 122 comprises a PTC heater element 136, it is preferable that the controller is configured to control the voltage applied across the PTC heater element 136. The amount of electrical energy converted to the thermal energy is determined by the difference between the operating temperature of the PTC heater and the actual temperature of the PTC heater. This is dependent on the water feed rate and gas flow rate.

The heater element 136 can operate in a temperature range from about 20° C. to about 500° C. More preferably the heater element 136 can operate in a temperature range of about 60° C. to 200° C. Even more preferably the heater element 136 can operate in a temperature range of about 120° C. to 150° C. If the heater element 136 is a PTC heater element, the heater element 136 can operate in a temperature range from about 20° C. to about 300° C., and if the heater element 136 is a non-PTC heater element, the heater element 136 can operate in a temperature range from about 20° C. to about 500° C. If the operating temperature of the heater element (or heater more generally) is at a lower temperature range (such as from about 20° C. to about 100° C. for example), then it has been found that a greater amount of the foraminous material in the heat conductor is preferred to achieve the desired heat transfer rate. Using lower temperatures might be preferable from an energy and safety perspective. On the other hand, if the operating temperature range of the heater element (or heater more generally) is at a higher temperature range (for example, from about 100° C. to about 300° C. for PTC heater elements in thermal contact with a heat conductor, or from about 100° C. to about 500° C. for non-PTC heater elements), then less foraminous material is required to achieve the desired heat transfer rate. Therefore, if a more compact humidifier is desired, then a higher operating temperature is preferable. A skilled person would recognise that the heater element 136 may operate at a different temperature, or different temperature range not specified above.

2.2 Foraminous Heat Conductor

The foraminous heat conductor 124 is used to vaporise water (from the fluid feeder 121) for humidification.

The foraminous heat conductor 124 preferably comprises an open cell metal foam. The metal foam 124 helps minimise the Leidenfrost effect, which increases the speed of vaporisation and assists in preventing aerosolization of water. The metal foam 124 helps to break water surface tension, which improves the extent that the dripped water spreads over the metal foam 124, or improves the extent that the dripped water “wets” the metal foam. This, combined with the large surface area that the porosity of the metal foam 124 provides relative to the water droplet provides an improved rate of heat transfer to the water, resulting in faster vaporisation of the water. The metal foam 124 has an additional effect of providing a distributed heating surface above the heating element 122, improving the vaporisation of any aerosols to the extent that aerosolization may occur. Preferably, the material selected in the metal foam 124 is a foam with a thickness of about 1.5 mm. However more generally, the metal foam should be thick enough to carry enough water such that bubbles in the water can be contained in the foam. This is because when water contacts a hot surface and boils, bubbles form. In order to reduce the likelihood of aerosols being formed under all operating conditions, the foam should be thick enough that these bubbles are wholly contained within the foam. A thickness in the range of 1-6 mm could be suitable, but other thickness ranges could be suitable as long as thickness of the foraminous heat conductor 124 is thick enough to carry the water/contain bubbles, yet thin enough to reduce thermal mass and cost.

The foraminous heat conductor 124 can operate in a temperature range from about 20° C. to about 500° C. More preferably the foraminous heat conductor 124 and/or heater 122 can operate in a temperature range of about 60° C. to 200° C. Even more preferably the foraminous heat conductor 124 and/or heater 122 can operate in a temperature range of about 120° C. to 150° C. If the foraminous heat conductor 124 is in thermal contact with a PTC heater element, the foraminous heat conductor 124 can operate in a temperature range from about 20° C. to about 300° C., and if the foraminous heat conductor 124 is in thermal contact with a non-PTC heater element, the foraminous heat conductor 124 can operate in a temperature range from about 20° C. to about 500° C. If the operating temperature of the heat conductor (or heater more generally) is at a lower temperature range (such as from about 20° C. to about 100° C. for example), then it has been found that a greater amount of the foraminous material in the heat conductor is preferred in order to achieve the desired heat transfer rate. Using lower temperatures might be preferable from an energy and safety perspective. On the other hand, if the operating temperature range of the heat conductor (or heater more generally) is at a higher temperature range (for example, from about 100° C. to about 300° C. for PTC heater elements in thermal contact with a heat conductor, or from about 100° C. to about 500° C. for non-PTC heater elements), then less foraminous material is required to achieve the desired heat transfer rate. Therefore, if a more compact humidifier is desired, then a higher operating temperature is preferable. A skilled person would recognise that the foraminous heat conductor 124 and/or heater 122 may operate at a different temperature, or different temperature range not specified above.

2.3 Fluid Feeder

The fluid feeder 121 refers to the means for supplying water to the foraminous heater 120 for humidification.

Preferably the fluid feeder 121 comprises a pump 126 that supplies water to the foraminous heater 120 through the fluid feeder 121. The pump 126 can be considered separate to the fluid feeder 121, or alternatively the pump 126 can be considered a component that the fluid feeder 121 comprises, as context allows. The pump 126 is controllable by a controller 129 that can accurately control the amount of water delivered to the foraminous heater 120. The controller 129 can receive the ambient humidity and current gas flow as an input to determine the water flow that the pump 126 should actuate at to achieve the desired humidity. The controller 129 can be configured to determine and control the appropriate amount of water that the pump 126 delivers to the foraminous heater 120 based on the flow rate of gas being delivered to the patient 18. The pump 126 injects water through an aperture 146 of the fluid feeder 121. The water drips onto the foraminous heat conductor 124, by falling under gravity. Alternatively, the tip of the fluid feeder 121 can be closer to, or in contact with the foraminous heat conductor 124, and water can be fed directly onto the foraminous heat conductor 124. The fluid feeder 121 is configured such that if the desired humidity is known, and if the current flow of gas is known, the fluid feeder (preferably by using the controller 129 to control the pump 126) can adjust the flow of water rapidly such that the desired humidity is achieved within seconds of inputting the desired humidity into the controller 129.

2.4 Overflow Drains

If the water reservoir 128 is located below the tub 118, a tub overflow drain 134 can be provided at the base of the tub 118. If there is a fault with the rate that water is fed by the fluid feeder 121 onto the foraminous heater 120, the tub overflow drain 134 can drain excess water into the water reservoir 128. The provision of a tub overflow drain 134 may be used to manage any vaporise water condensed on the walls of the mixing chamber 112 or tub 118, when conditions result in the relative humidity in the mixing cavity to exceed 100% (e.g. the relative humidity within the mixing chamber 112, (including in the tub 118) exceeds 100%). Over time, this condensation will agglomerate into droplets which will flow down the sides of the chamber, and ultimately down the drain when there is a large enough volume of condensed water. This is one way to lower the risk of liquid water travelling to the patient.

Similarly, if the water reservoir 128 is located below the mixing cavity 112, a mixing cavity overflow drain 135 can be provided at the base of the mixing cavity 112. The provision of a mixing cavity overflow drain 135 may be used to manage any vaporise water condensed on the walls of the mixing chamber 112 or tub 118, when conditions result in the relative humidity in the mixing cavity to exceed 100% (e.g. the relative humidity within the mixing chamber 112, (including in the tub 118) exceeds 100%). Over time, this condensation will agglomerate into droplets which will flow down the sides of the chamber, and ultimately down the drain when there is a large enough volume of condensed water. This is another way to lower the risk of liquid water travelling to the patient.

### 3. Second Embodiment

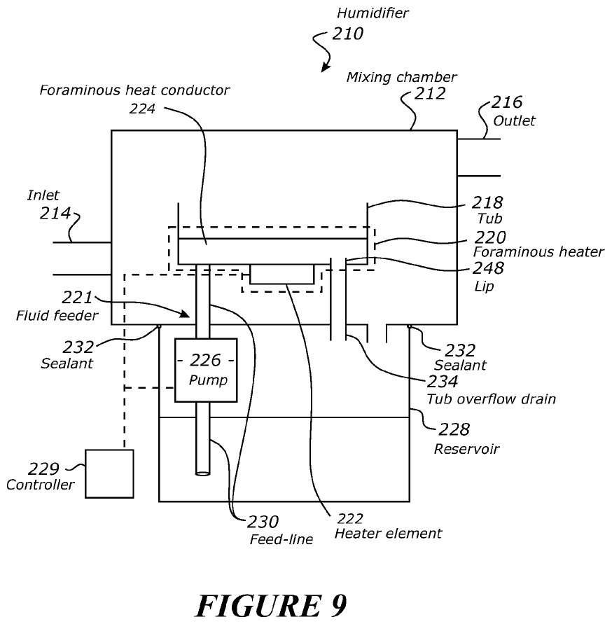

FIG. 9 shows a second embodiment of a humidifier 210. The humidifier 210 has similar features and functions already described in the first embodiment, but with some differences as will be described. Unless described otherwise, the features numerically labelled on FIG. 9 are equivalent in function to the respective features described in the first embodiment. For example, item 212 relates to a mixing chamber. Discussion of the second embodiment therefore focuses on how this embodiment may differ from the first embodiment.

One difference is that the humidifier 210 comprises a tub 218 which inside contains the foraminous heat conductor 224, but not the heater element 222. The heater element 222 is instead placed underneath the base of the tub 218. The heater element 222 may be a separate component, may be fixed to the base of the tub 218, or may be fixed to the base of the mixing chamber 212 (but thermally insulated therefrom). The foraminous heater 220 (shown nominally in dotted lines) is formed when the heater element 222 and the foraminous heat conductor 224 are placed such that they are in thermal contact with each other. Preferably, this means positioning the tub 218 in the appropriate location (which could be a thermally isolated location from the mixing chamber) within the mixing chamber 212.

Another difference is that both the heater element 222 and the foraminous heat conductor are oriented horizontally, as opposed to vertically in the first humidifier embodiment 110.

Another difference is that the humidifier 210 comprises a fluid feeder 221 that feeds (as opposed to dripping) water into the bottom of the tub 218 (preferably by injection of water), and therefore feeds water into the bottom of the foraminous heat conductor 224. The pump 226 supplies water to the base of the tub 218. In operation, the pump 226 supplies water at a low flow rate. Feeding water from underneath the foraminous heat conductor 224 provides sufficient local pressure to wet the foam 224, overcoming variability in foam-water contact angle due to temperature, water mineral deposits, and organic compounds adsorbed from the air, and any other variables. Further, supplying water to the base of the foam 224 in this manner may help to further reduce any spatters and aerosol that may be generated if water is dropped onto the foam 224.

Another difference is that a lip 248 may be provided on top of the tub overflow drain 234 so that a layer of water formed along the bottom of the tub 218 can still “wet” the foraminous heat conductor, while allowing excess water to drain back into the reservoir.

### 4. Third Embodiment

FIG. 10 shows a third embodiment of a humidifier 310. The humidifier 310 has similar features and functions already described in the earlier embodiments, but with some differences as will be described. Unless described otherwise, the features numerically labelled on FIG. 10 are equivalent in function to the respective features described in earlier embodiments. For example, item 312 relates to a (conduit) mixing chamber.

This humidifier 310 is an in-conduit embodiment that allows for use inside the conduit. The humidifier 310 may be independently positioned anywhere within a conduit, including positioning the humidifier 310 proximal to the patient 18. The components described in this embodiment allow for a humidifier 310 that is smaller than the earlier described humidifiers 110, 210. The in-line humidifier 310 may be easier to manufacture.

The humidifier 310 has a mixing chamber 312 that is shaped as a conduit portion, or be placed in a conduit-shaped housing. The conduit portion 312 houses a foraminous heater 320, in which the foraminous heater 320 houses a heater element 322 and a foraminous heat conductor 324. The foraminous heater element 320 is a spiral that forms a coil, and the foraminous heat conductor 324 is an outer cylindrical body of ceramic or metallic foam 324 that wraps around the heater element 322. Gas flows in from the inlet portion 314 of the conduit 312, through the heater element 322 and around the metal foam 324. Gas then flows to the outlet portion 316 of the conduit 312. As part of a fluid feeder 321, a pump 126 pumps water from a water reservoir and can feed water directly (rather than dripping, to avoid any water spatter and droplets forming) onto the foam 324 wrapped around the heater element 322.

The heater element 322 may be battery powered 350 (AC, DC, or both). The heater element 322 may comprise of PTC material, but alternatively, the heater element 322 may not comprise of PTC material. Discussion will first turn to a variant where the heater element 322 comprises a PTC material.

The heater element 322 can comprise PTC material, including a PTC ceramic material. As explained earlier, the PTC material in the heater element 322 allows the operating temperature of the heater element 322 to be self-regulated. The heater element 322 does not require a temperature feedback loop controller in this instance. However, a skilled person would also recognise that other control mechanisms (of which controller 329 can be a component of for example) may optionally be implemented in addition to the inherent temperature and power control provided by the PTC material of the PTC heater element, although this is not essential. For example, there may optionally be feedback loops that monitor and/or control electrical resistance or humidity if the appropriate sensor/s and circuitry is provided.

Discussion now turns to another variant of the heater element 322 in which the heater element does not comprise PTC material (i.e. a non-PTC heater element). In a non-PTC heater element, the heater element 322 will have an electrical resistance that does not increase exponentially with an increase in operating temperature. Consequently, a feedback loop is required for temperature control of the heater element 322, and for maintaining safe operating temperature limits. This also means sensors are used to monitor the temperature of the non-PTC heater element, whether sensing directly or indirectly. As an example, a feedback loop for ensuring safe operation of the heater element 322 could come in the form of controlling the rate that water is supplied to the foam 324. The electrical potential across the heater element 322 can be sensed and measured by a voltage sensor 327, which in this example is an uninsulated wire that wraps around the foraminous heater 320 like a voltage sensing ring for example. If an insufficient amount of water is pumped to the foam 324, the heater element 322 (that the foam 324 is thermally connected to) will be excessively hot, causing an increase in electrical potential across the foam 324, and will be sensed by the voltage sensor 327. A relatively high voltage reading from the voltage sensor 327 causes the controller 329 to control the pump 326 to pump a greater flow of water to the foam 324. The additional flow of water delivered to the foam 324 helps in cooling the temperature of the heater element 322. Another example of a feedback loop could be to control the relative humidity output as a result of a direct measurement of humidity output. Another example of a feedback loop could be to control the gas flow rate. Another example of a feedback loop could be to control the power provided to the heater element 322, based on the proportional relationship between the power provided and the temperature of the heater element 322. A skilled person would recognise that there are other ways of implementing a feedback loop to regulate the operating temperature of the heater element 322. A skilled person would also recognise that feedback loops (including sensor/s) as discussed are to be taken into account when implementing any (other) humidifier embodiment with a non-PTC heater element. Although the feedback loops as described are used as part of a non-PTC heater element, a skilled person would appreciate that the feedback loops as described may optionally be used with a PTC heater element, but not essential as the PTC material of the PTC heater element provides inherent temperature and power control.

### 5. Fourth Embodiment

FIG. 11 shows an end-on view of a fourth embodiment of a humidifier 410. The humidifier 410 has similar features and functions already described in the earlier embodiments, but with some differences as will be described. Unless described otherwise, the features numerically labelled on FIG. 11 are equivalent in function to the respective features described in earlier embodiments. For example, item 412 relates to a (conduit) mixing chamber. Discussion of the fourth embodiment focuses on how this embodiment may differ from the earlier embodiments, and in particular, the third embodiment.

The humidifier 410 can be configured in the same way as what has been described for humidifier 310. However, one difference between humidifier 310 and humidifier 410 is the difference in the fluid feeder 421, which has no pump. Instead the supply of water is inherently controlled using one of: a pressurised cartridge of water, differential capillary action, or by using differential osmotic potential to draw water out from the water reservoir 428 to the foraminous heat conductor 424 (which may be a metal foam).

### 6. Alternative Embodiments

Discussion below now turns to alternative embodiments that the inventors have envisioned. The embodiments that will be described below can exist as an embodiment separate to the embodiments already discussed, or they can be seen as additional modifications to the embodiments already discussed. The alternative embodiments below are discussed by way of example only, however a skilled person would understand that other embodiments may also exist by making the appropriate modifications commonly known in the art.

6.1 Heater Element

The heater element can be mounted vertically (as shown in FIG. 6) or horizontally (FIG. 9). The way in which the heater element is oriented influences the way that the water wets, flows, or is distributed within the foraminous heat conductor.

There can be more than one heating element, either in parallel tubs, or there can be multiple heater elements in the same tub. The heater elements can be connected to the same, continuous contact heater. Alternatively, the heater elements can all operate independently of each other.

If the heater element is a PTC heater element, temperature of the PTC heating element can be varied, for example, from about 20° C. to about 300° C. The PTC heating element can be a ceramic, or another type of PTC material, such as a polymer. The power supply to the PTC heating element can be low voltage, e.g. from about 6V to about 48V DC, or mains voltage, e.g. from about 100V AC to about 230V AC.

Components of the heater element can be molded into the base of the tub. Alternatively, the tub can be overmolded onto the heater element and/or overmolded onto the thermal insulator (if present). Alternatively, the heater can be permanently or removably connected to the tub, using a snap-fit, friction fit or interference fit, or the like, to connect the components. The heater element can be connected to the tub via the thermal insulator.

6.2 Foraminous Heat Conductor

The foraminous heat conductor can be a nickel, copper or stainless steel metal foam, an alloy of any of those metals, or be any other relatively inert metal. Alternatively, it could be a non-metallic high temperature open cell foam or sponge, such as a thermally conductive plastic or a porous ceramic for example.

As opposed to an open cell metal foam, the foraminous heat conductor can be extruded with a foaming agent, deep pressed, machined, sintered, or fabricated by another manufacturing method to create a foraminous heat conductor with a relatively high surface area. The surface of the foraminous heat conductor can be treated or coated. The surface of the foraminous heat conductor can be treated or coated with a hydrophilic material to encourage the spread of water over a large surface area. The foraminous heat conductor may be treated or coated to prevent or reduce oxidation.

The foraminous heat conductor can be wire wrapped, glued, clipped, clamped, bonded to a frame, or otherwise affixed to the heater element.

As an alternative to the metal foam, the foraminous heat conductor can be, or further comprise a high density metal mesh.

The foraminous heat conductor can be an interchangeable, replaceable component.

6.3 Foraminous Aerosol Filter

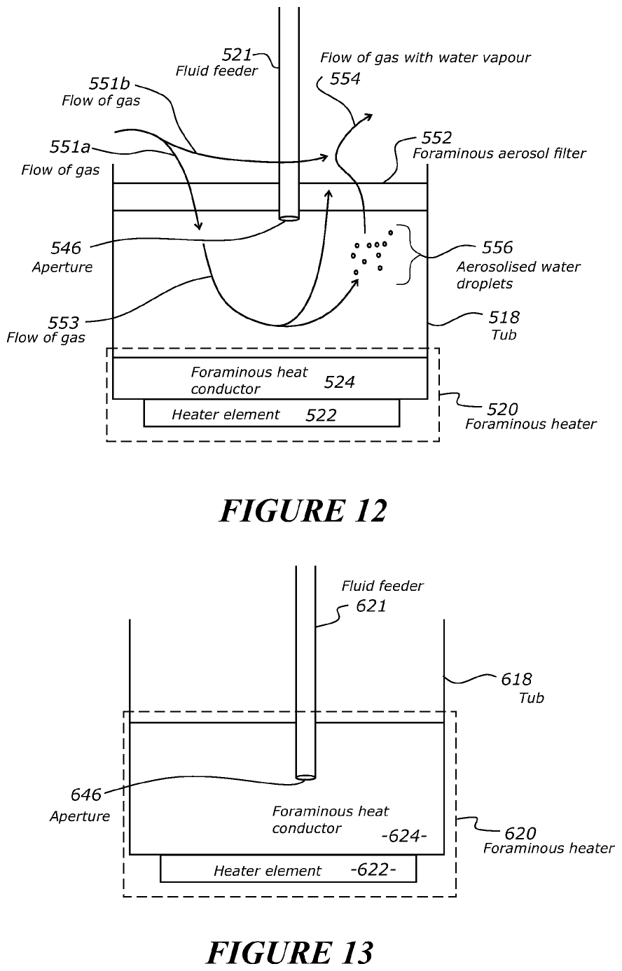

In a situation where aerosolised water droplets might be formed, a foraminous aerosol filter can be used to prevent aerosols from escaping from the tub. One example is illustrated in FIG. 12. In this example, the foraminous aerosol filter 552 is in thermal contact with the foraminous heater 520, which allows the foraminous aerosol filter 552 to vaporise any aerosolised droplets 556 formed within the tub 518. In use, an incoming flow of gas 551a flows through the foraminous aerosol filter 552 to enter the tub 518. Alternatively, gas may flow 551b across the top of the tub 518. The vaporised water humidifies the flow of gas 553 in the tub 518. The flow of gas 554 then escapes the tub. The foraminous aerosol filter 552 allows the vaporised water to leave the tub as part of the exiting flow of gas 554. The water vapor 554 that escapes from the tub 518 also mixes with the flow of gas 551b in the mixing chamber for humidification. At the same time, the foraminous aerosol filter 552 vaporises any remaining aerosolised droplets 556 within the tub 518 to form part of the exiting flow of gas 554. Any flow passing through the foraminous aerosol filter 552 and out of the tub 518 is heated. In some situations, the aerosolised droplets 556 may condense on the wall of the tub 518. When this happens, the condensed water droplets 556 may be accumulate and fall/drip onto the foraminous heat conductor 524 which in turn causes vaporisation of the water droplets 556. In the illustrated embodiment, the foraminous aerosol filter 552 is a heat conductor that is thermally connected to a heat source. In the example of FIG. 12, the tub 518 is metallic (but could alternatively comprise a thermally conductive non-metallic material) so the foraminous aerosol filter 552 and the foraminous heater 520 are in thermal contact with each other. However, a skilled person would recognise that there are other ways for the foraminous aerosol filter 552 and the foraminous heater 520 to be in thermal contact with each other, and using a metallic tub is simply one of many ways to achieve this. For example, the foraminous aerosol filter may instead be in thermal contact with a heat source operating independently of the foraminous heater 520.

6.4 Fluid Feeder

The fluid feeder used in the embodiments can be an active system, or a passive system. For example, it can be driven by an electric pump, an electro-osmotic pump, a gravity feed system (including at least one control valve for example), breathing circuit pressure, suction pressure (generated by a venturi located along the gas flow path), or capillary pressure.

Water flow can be measured by a thermal mass flow sensor, or inferred indirectly (for example the overfeeding of water can be inferred by reading the temperature of the heater element, or by reading the humidity within the tub). Additionally, the water flow actuated by the pump may be controlled by calling up running speed, either automatically and/or manually. These measures prevent water from accumulating and overflowing out of the tub.

As shown in FIG. 13, the fluid feeder 621 could supply water to the foraminous heat conductor 624 through an aperture 646 located at the bottom of the fluid feeder 621, directly into the foraminous heat conductor 624. Water can be injected through the aperture 646 into any part of the foraminous heat conductor 624 such that the spattering of water is minimised when injected. Alternatively, the fluid feeder could supply water to the foraminous heat conductor at a distance from the foraminous heat conductor.

There could be a liquid water sensor (not shown) in the tub. When activated, the liquid water sensor would prompt the controller to reduce the water pump rate. Alternatively, an algorithm can be programmed into the controller, taking input variables such as available power, flow rate, temperature inputs, and the like.

6.5 Tub

Some alternative embodiments of the tub are envisioned (as alternative to the configuration shown in earlier embodiments, where gas passes into the top of the tub and out of the top):
Gas could enter from the bottom of the tub and exit through the top.
The gas could be directed to spiral through the tub with a helical spiral configuration.
Gas could enter from the side of the tub and exit from the side of the tub.

To minimise environmental heat loss and condensation build-up inside the humidifier, the tub and/or the mixing chamber may be double walled or otherwise insulated. Additionally, the air could be pre-heated prior to reaching the humidifier to reduce condensation build-up. The tub and mixing chamber may be combined into a single chamber.

6.6 Method of Humidifying Gases

There can be a method of humidifying a flow of gases using a humidifier comprising a heater. This involves heating the heater, feeding a flow of gases into the humidifier, and feeding fluid to the heater such that fluid vaporises upon contact with the heater. The heater can have a heat conductor, preferably comprising foraminous material, and fluid can be dripped onto the heat conductor.

One or more of a fluid reservoir, a pump, and a feed-line can be used to feed water onto a heater of a humidifier. This involves operating a pump to pump fluid from a fluid reservoir through a feed-line, and feeding fluid to the heater. The flow of fluid fed to the heater is fed at an adjustable flow rate, in which the flow rate is determined based on desired humidity and/or flow of gas through the humidifier. In some embodiments, the act of feeding fluid to the heat conductor (or heater) involves dripping fluid onto the heat conductor (or heater).

There can be a method of heating a humidifier comprising a heater element, and a heat conductor. This involves operating the heater element, which generating heat, as well as conducting heat from the heater element to the heat conductor. The heater element can comprise PTC material, and the heat conductor can be foraminous.

A controller can also be implemented to perform any one of the method steps described in this method embodiment, unless context dictates otherwise.

Any of the earlier described medical gases delivery system embodiments and/or earlier described humidifiers embodiments can be used for a method of humidifying a flow of gas and/or for a method of heating a humidifier. This can also include any combination of preferred/optional features that can be added onto any of the earlier described medical gases delivery system embodiments and/or earlier described humidifiers embodiments.

6.7 Application of the Embodiments

Any of the earlier described medical gases delivery system embodiments and/or earlier described humidifiers embodiments can be used in any situation where humidification is desired. This can also include any combination of preferred/optional features that can be added onto any of the earlier described medical gases delivery system embodiments and/or earlier described humidifiers embodiments.

### 7. Possible Features of the Embodiments

1. A tub for a humidifier, comprising:
a body for humidification
a first heat conductor and a second heat conductor arranged to form a humidification region and/or gas flow path in the body between the first heat conductor and the second heat conductor, and
wherein the second heat conductor is foraminous to allow gas to pass to and from the humidification region and/or gas flow path.

2. A tub according to clause 1, wherein the second heat conductor has voids in a solid structure.

3. A tub according to clause 1 or 2, wherein the foraminous heat conductor is foam.

4. A tub according to clause 3, wherein the foam is open cell foam.

5. A tub according to clause 3 or 4, wherein the foam is formed from metal or ceramic.

6. A tub according to any one of clauses 1 to 5, wherein the tub is adapted to receive an injection of water.

7. A tub according to clause 6, wherein the tub is adapted to receive the injection of water from a fluid delivery apparatus.

8. A tub according to any one of clauses 1 to 7, wherein the tub is adapted to be installed in a humidifier with a heater element to heat the first and/or second heat conductor.

9. A tub according to clause 8, wherein the heating element comprises a positive temperature coefficient (PTC) material.

10. A tub for a humidifier according to any of clauses 1 to 9, wherein the humidifier is:
a standalone humidifier for connection to a flow of gas
a humidifier for removable connection to a flow source
a humidifier forming part of a flow source.

11. A humidifier for humidifying a gas, comprising:
a body for humidification
a first heat conductor and a second heat conductor arranged to form a humidification region and/or gas flow path in the body between the first heat conductor and the second heat conductor, and
a heating element for heating the first and/or second heat conductor, wherein the second heat conductor is foraminous to allow gas to pass to and from the humidification region.

12. A humidifier according to clause 11, wherein the second heat conductor has voids in a solid structure.

13. A humidifier according to clause 11 or 12, wherein the foraminous heat conductor is foam.

14. A humidifier according to clause 13, wherein the foam is open cell foam.

15. A humidifier according to clause 13 or 14, wherein the foam is formed from metal or ceramic.

16. A humidifier according to any one of clauses 11 to 15 further comprising a fluid delivery apparatus for injecting fluid onto the first and/or second heat conductor.

17. A humidifier according to clause 16, wherein the first and/or second heat conductor is adapted to substantially vaporise fluid injected by the fluid delivery apparatus when fluid contacts the first and/or second heat conductor.

18. A humidifier according to any of clauses 11 to 17, wherein the humidifier is:
a standalone humidifier for connection to a flow of gas
a humidifier for removable connection to a flow source
a humidifier forming part of a flow source.

19. A humidifier according to any of clauses 11 to 18, wherein the second heat conductor is adapted to vaporise aerosolised fluid when aerosolised fluid contacts the second heat conductor.

20. A humidifier according to any one of clauses 11 to 19, further comprising a tub.

21. A humidifier according to clause 20, wherein the first and second heat conductors are disposed within the tub.

22. A humidifier according to any one of clauses 11 to 21, wherein the first and/or second heat conductor is foraminous.

23. A humidifier according to clause 22, wherein the first and/or second heat conductor has voids in a solid structure.

24. A humidifier according to clause 22 or 23, wherein the first and/or second heat conductor is foam.

25. A humidifier according to clause 23, wherein the foam is open cell foam.

26. A humidifier according to clause 24 or 25, wherein the foam is formed from metal or ceramic.

27. A humidifier according to any one of clauses 22 to 26, wherein the first and/or second heat conductor is a metal foam.

28. A humidifier according to claim any one of clauses 11 to 27, wherein the first heat conductor is thermally connectable to the heating element.

29. A humidifier according to any one of clauses 11 to 28, wherein the heating element comprises a positive temperature coefficient (PTC) material.

30. A humidifier according to any one of clauses 11 to 29, wherein the heating element self-regulates the upper limit of its temperature.

31. A humidifier according to any one of clauses 11 to 30, wherein the humidifier is:
a standalone humidifier for connection to a flow of gas
a humidifier for removable connection to a flow source
a humidifier forming part of a flow source

1. A humidifier, comprising:
a gas flow path, and
a heater in the gas flow path, the heater comprising:
a heater element, and
a heat conductor in thermal contact with the heater element,
wherein the heat conductor is foraminous.

2.-75. (canceled)

76. The humidifier of claim 1, wherein the heat conductor is a foam.

77. The humidifier of claim 76, wherein the foam is open cell foam.

78. The humidifier of claim 1, wherein the heat conductor comprises metal foam or ceramic foam.

79. The humidifier of claim 1, wherein the heat conductor is configured to:
ameliorate a Leidenfrost effect; and/or
increase a rate of heat transfer.

80. The humidifier of claim 1, wherein the heater element comprises PTC material.

81. The humidifier of claim 1, wherein the heater is located within a conduit.

82. The humidifier of claim 1, wherein the humidifier has an overflow drain.

83. The humidifier of claim 1, further comprising a fluid delivery apparatus, wherein the fluid delivery apparatus is adapted to drip fluid onto the heat conductor.

84. The humidifier of claim 1, wherein the heater is in a humidifier tub.

85. The humidifier of claim 1, wherein the heater element comprises a PTC material, and the heat conductor comprises one or more of:
nickel,
ceramic,
copper; or
stainless steel.

86. The humidifier of claim 84, wherein the humidifier is configured to operate in a temperature range from about 100° C. to about 300° C.

87. The humidifier of claim 1, further comprising a water sensor coupled to the heater.

88. The humidifier of claim 1, wherein the heater element is a coil.

89. The humidifier of claim 1, wherein the heat conductor is wrapped cylindrically around the heater element.

90. The humidifier of claim 1, wherein the humidifier is configured to warm up in less than approximately one minute.

91. The humidifier of claim 1, wherein the heater element can be temperature controlled via a feedback loop.

92. A humidifier, comprising:
a gas flow path, and
a heater in the gas flow path, the heater comprising:
a heater element, and
a heat conductor in thermal contact with the heater element,
wherein the heater element comprises a PTC material.

93. A humidifier, comprising:
a gas flow path, and
a heater in the gas flow path, the heater comprising:
a heater element, and
a heat conductor in thermal contact with the heater element,
wherein the heater element comprises a PTC and a foraminous material.
